(19) United States
(12) Patent Application Publication
(10) Pub. No.: US 2015/0273154 A1
(43) Pub. Date: Oct. 1, 2015

(54) RECHARGEABLE AND RESTERILIZABLE MIXING DEVICE WITH PHYSIOLOGICAL GAS AND SOLUTION TO CREATE FOAM WITH MICROBUBBLES, USED IN ENDOVASCULAR TREATMENTS

(71) Applicant: Murillo Caporal Piotrovski, Florianopolis (BR)

(72) Inventor: Murillo Caporal Piotrovski, Florianopolis (BR)

(21) Appl. No.: 14/675,039

(22) Filed: Mar. 31, 2015

Related U.S. Application Data
(60) Provisional application No. 62/035,784, filed on Aug. 11, 2014.
(60) Provisional application No. 61/972,657, filed on Mar. 31, 2014.

(51) Int. Cl. A61M 5/31; A61M 5/19

(52) U.S. Cl. CPC A61M 5/31; A61M 5/19; A61M 2005/006

(57) ABSTRACT

A mixer to create foam with microbubbles using physiological gases currently used in sclerotherapeutic treatments of varicose veins has anatomical and compact dimensions and is built with material resistant to sterilization and pressure. The device is mounted on the body around an essentially cylindrical axle comprised of a central valve support, while the top conical body portion is crossed in its center by a flow orienting duct. This axle is coupled at the center of a spinning circular reservoir constituted by the body, containing several housings for the application of solutions with different concentration levels. These parts are locked by a connector at the bottom to the gas duct with a spray nozzle. Each housing has a channel that can be aligned to communicate with the inside of the flow orienting duct of the central axle, responsible for orienting the produced mixture to a top reservoir with a foam-making nozzle.

### TECHNICAL FIELD

The present invention relates to a rechargeable, resterilizable, portable, and easy-handling device to create high consistency foam using physiological gases and sclerosing agents; foam currently used in sclerotherapeutic treatments of varicose veins. It may be used also to create foam for other therapeutic purposes.

This reservoir has ergonomic and compact dimensions and is elaborated with material resistant to sterilization and pressure, designed to be used in treatments at doctor's offices, clinics or hospital centers, but can also be made of appropriate materials for single use applications.

### BACKGROUND OF THE INVENTION

As widely known, the endovascular treatment is carried out inside vessels in order to treat circulatory diseases in blood vessels, arteries, or veins.

Sclerotherapy is the name given to the method that consists of the injection of some product into varicose veins in order to sclerose them.

Today, a liquid widely used in this method is the hypertonic glucose. It is often used separately in a concentration of 75% or lower (50%), with the addition of another substance to increase its sclerosing capacity.

The foam is applied in this context, that is, in order to increase the sclerosis capacity. It consists of the mixing of a medication, currently Polydocanol or tetradecil-sulphate, mixed with regular air, or a physiological gas such as carbon dioxide, and then vigorously agitated to form a dense foam that, when injected in the varicose vein, remains in contact with the vessel walls for a longer period than just the liquid sclerosant, thereby increasing its sclerosing capacity and enabling the treatment of varicose veins larger than 0.4 cm in diameter.

In current treatments, a liquid or foam is used and referred to as sclerosants, which are injected through needles of several sizes, depending on the size of the vein to be treated. This liquid or foam causes an alteration in the blood vessel wall cells that later causes its occlusion. When the liquid or foam remains in the circulation, it is diluted by the blood and loses its concentration and effect.

The foam used today is produced in a homemade way, using syringes, a three-way key, regular air, and a liquid. This foam contains irregular, large bubbles, which dilute its density and cohesion. Consequently, this homemade foam dilutes more easily inside the blood stream and loses its function, which is to lesion the internal wall of the varicose vessel. That is, it is less effective when compared with foam with microbubbles.

In order to obtain the microbubbles, a chemical sclerosing foam of good quality has to be prepared with the sclerosant and physiological gases under properly calibrated pressure that, when injected in the vessel, provides better density and higher efficacy in the treatment of larger varices. The use of physiological gases allows physicians to use a higher quantity of foam in each treatment session with higher safety, providing better and quicker results in the proposed treatment. This new technique using foam with microbubbles is less invasive, does not require resting periods, and the patient does not have to interrupt its daily activities as the procedure is not surgical.

### PRIOR ART

Reservoirs or equipment intended to create foam are already known in several areas, such as pesticide reservoirs, fire extinguishers, shaving foam, and others. These containers have different sizes and characteristics that suit each functionality, with different means of recharge or creation of foam.

Today, professionals in the field are aware of the use of “three-way taps” in endovascular treatments to create homemade sclerosing foam adaptable directly to syringes, where the connection is done and the direction of the flow is controlled with three different lines: two infusion lines in “luer lock” female ends, and a third infusion line or venous access device in its male “luer slip” or “luer lock” connection. It also includes a knob that acts as flow shutter and switch.

Patent application BR 0407003-8, filed on Aug. 19, 2004, describes a foam forming unit comprised of: a mixing chamber (12) that communicates with the output (14) of a pump in order to mix liquid and air, a distribution part (22) equipped with an output flow channel (24) with a foam opening (26) to distribute foam, where the output flow channel has communication with the mixing chamber (12) and the first foam forming element (28) placed in the output flow channel, so that the foam that flows through the output flow channel passes through the foam forming element (28) at least twice, where the distribution part is also equipped with a nozzle element (51) that includes at least a final part of the output flow channel and the foam opening, where the output flow channel includes a cavity (32) after the first passage through the first foam forming element, with this cavity positioned before the nozzle element, as observed in the flow direction.

Patent application BR 0414281-0, filed on Sep. 9, 2004, describes a foam transfer device (600) to be used with an aerosol containing device to produce sclerosing foam for the treatment of varicose veins, among other things. This device enables the deviation of an initial quantity of foam with the specification below from the container to be dispensed, for instance, into a full dispensing changer before releasing an additional quantity of foam to be used in treatments. The flow switching from the dispensing chamber to a different output (618) for use is done without interruption in the flow from the aerosol container, as this causes the foam to go down under specification again. The dispensing chamber may be transparent, so that the foam entering it can be observed, allowing the user to decide when to stop sending foam to be dispensed. Alternatively, the foam can be deviated automatically, for instance, at the end of a preset time or present volume of foam to be dispensed. The foam is usually released into a syringe for injection into a varicose vein of the patient.

In view of the techniques and devices found in the current state of art, the applicant proposes an unprecedented mixing device used to create sclerosing foam with microbubbles, also introducing advantages and improvements to the deficiencies found in the current techniques and models.

### BRIEF DESCRIPTION OF THE FIGURES

For better visualization and understanding of the object intended to be protect by this patent application, the object will be described below with the aid of the attached figures

FIG. 1 illustrates a side view of the mixing device in a first embodiment, as held by the hand of a professional;

FIG. 2 illustrates a side view of the mixing device in a second embodiment, as held by the hand of a professional;

FIG. 3 shows a top view of the device in its first embodiment, indicating the cross section A-A;

FIG. 4 shows the cross sectional view A-A indicated in the previous figure;

FIG. 5 shows a cross sectional view of the device in the previous figure being used;

FIG. 6 shows an exploded view of the device in its first embodiment, according to the previous figure;

FIG. 7 shows a top view of the device in its second embodiment, indicating the cross section B-B;

FIG. 8 shows the cross section B-B indicated in the previous figure with the device being used;

FIG. 9 shows an exploded view of the device in its second embodiment, according to the previous figure;

FIG. 10 shows an exploded view of the device in a third embodiment;

FIG. 11 shows an exploded view of the device in a fourth embodiment;

FIG. 12 shows an exploded view of the device in a fifth embodiment;

FIG. 13 shows an exploded view of the device in a sixth embodiment;

FIG. 14 shows an exploded view of the device in the sixth embodiment, including a solution reservoir;

FIG. 15A shows a partially exploded cross-sectional view of the device in a seventh embodiment;

FIG. 15 B shows a partially exploded cross-sectional view of the device in an eighth embodiment;

FIG. 15C shows a cross-sectional view of the device in a ninth embodiment;

FIG. 15D shows a cross-sectional view of the device in a tenth embodiment;

FIG. 15E shows a cross-sectional view of the device in an eleventh embodiment;

FIG. 15F shows a partially exploded cross-sectional view of the device in a twelfth embodiment; and

FIG. 15G shows cross-sectional view of the device in a thirteenth embodiment.

### DETAILED DESCRIPTION AND ADVANTAGES OF THE DEVICE

This patent application is explained in details in accordance with the attached figures.

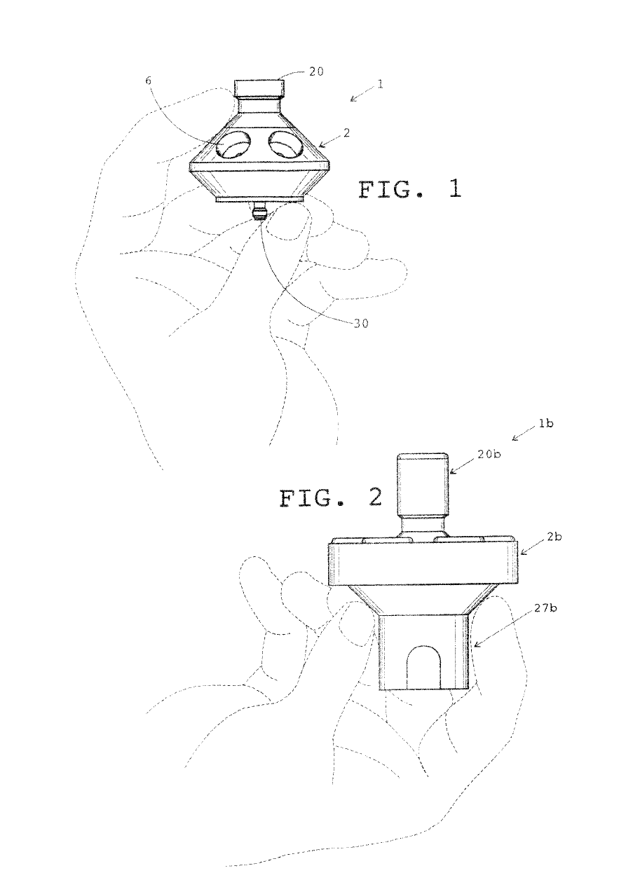

FIG. 1 shows an embodiment of the object of this patent application with a front view of the rechargeable, resterilizable mixing device, with physiological solution and gas to create foam with microbubbles used in endovascular treatments. FIG. 2 shows a different embodiment from the object shown in FIG. 1.

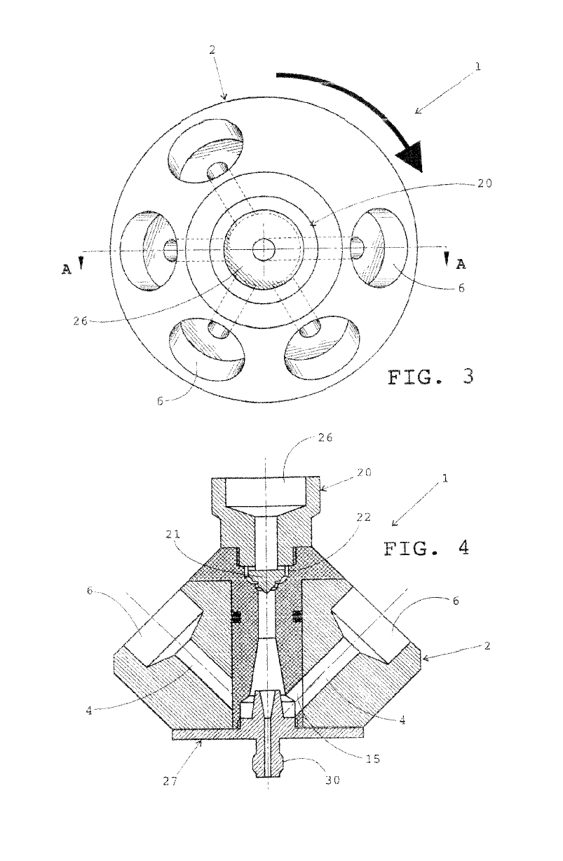

According to FIGS. 3 to 6, the mixing device (1) claimed in this patent application comprises a body (2) with the shape of two inverted cones joined by their bases, a central cylindrical channel (3) and inclined downward channels (4) that intercept the central cylindrical channel (3) at its bottom end (5). The inclined downward channels (4) include a cylindrical recess (6) in its top end to receive the syringe body by coupling.

The mixing device (1) also includes a central valve support (7), a cylindrical base (9), and a head (8) in the shape of a cone. The base (9) of the central support (7) has a sealing ring (10) on the outer surface and an internal opening (11) inside it, which crosses the central valve support (7) throughout its length. This opening (11) has a cylindrical recess (12) in its bottom end with an upward conical protrusion (13) that reaches half the length of the central support (7). Between the conical protrusion (13) and the cylindrical recess (12) there is a conical recess (14) that includes an inclined through hole (15), which enables the communication between the internal opening (11) with the external cylindrical surface of the central support (7). Above the conical protrusion (13) there is a cylindrical channel (16) that communicates with the head seat opening (17), located in the region of the head (8) of the central support (7). This head seat opening (17) includes conical housing recesses (18) in which the conical tip of the head (21) seats to form small passing openings (22) for the foam with microbubbles.

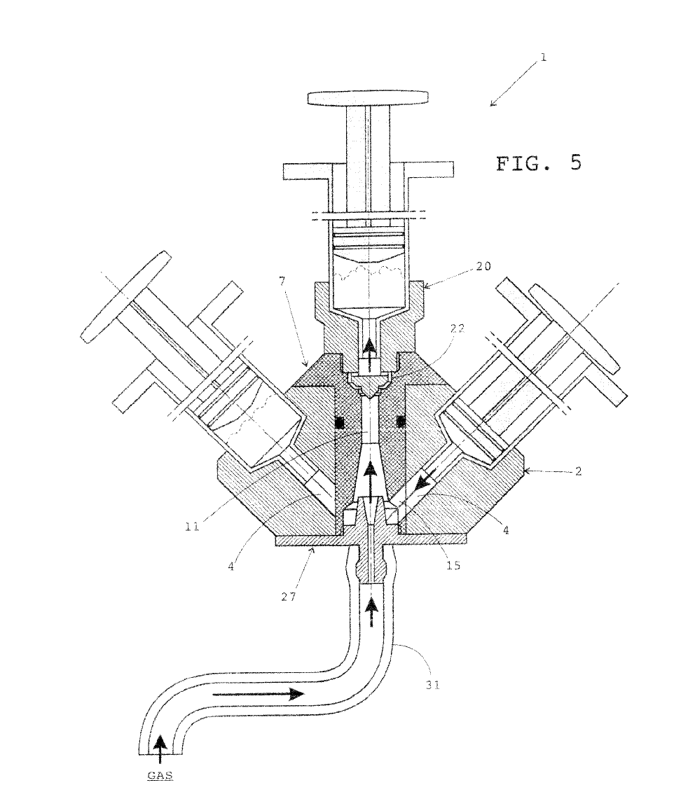

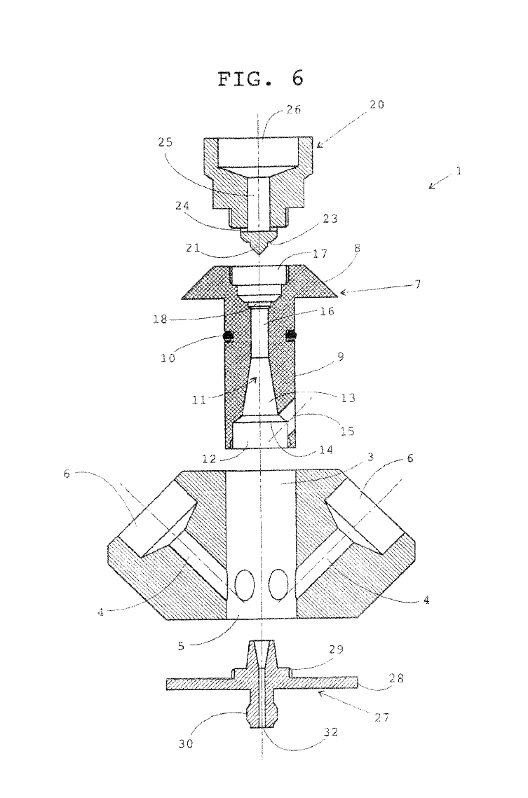

The head (20) is comprised of a cylindrical part with a tip (21) of staggered conical shape that forms a step (23) in its middle region to enable the formation of the passing channel (22) of the foam with microbubbles (FIGS. 4 and 5). Above the tip (21) there are openings (24) that communicate with the luer channel (25) that receives the external part of the syringe to be fitted into the orifice (26) of the head (20).

At the bottom of the device (1) a connector (27) is connected and comprised of a thin cylindrical disc (28) that is supported by the bottom part of the body (2) and couples into the internal opening (11) of the central valve support (7) by means of a threaded protrusion (29). This connector (27) has a fitting nozzle (30) in its bottom part to receive a hose (31), through which a gas enters and flows into the internal opening (11) of the central valve support (7) through the passage (32).

FIGS. 3, 4, and 5 explain the operation of the mixing device (1). The arrow in FIG. 3 indicates the direction of rotation of the body (2) to which the syringes shown in FIG. 5 are coupled. These syringes contain several solutions with different concentration levels to form the foam with microbubbles. It is possible to observe that each inclined downward channel. (4) in the body (2) communicates with the internal opening (11) through the inclined passing hole (15) of the central valve support. Each rotation of the body (2) presents a single inclined downward channel (4) that communicates with the through hole (15). Therefore, only a given syringe containing a solution with a given concentration level will have fluid communication with the opening (11) and consequently with the passage of gas (32) and luer channel (25). The gas flow causes the solution to flow through the narrow passages (22), enabling the formation of the microbubbles in the foam that enter the syringe inserted into the head (8). Alternatively, to assist in the formation of microbubbles, a small sponge of micro-porous material can be placed in the luer channel (25).

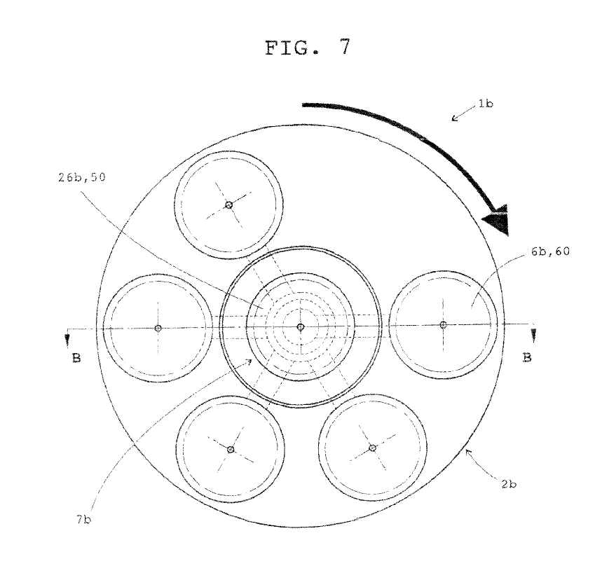

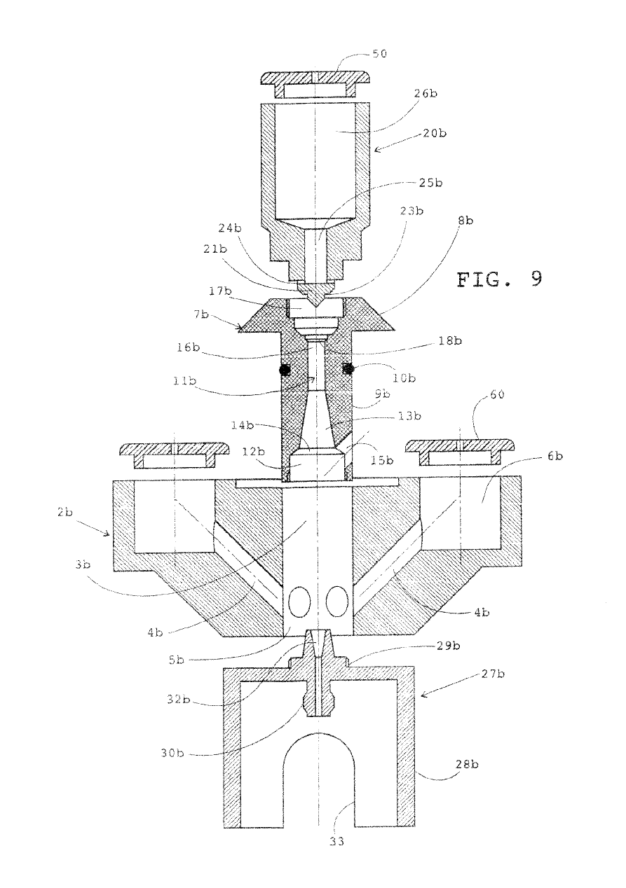

FIGS. 7, 8, and 9 show an embodiment of the object of the rechargeable, resterilizable mixing device with physiological solution and gas to create foam with microbubbles used in endovascular treatments. In this embodiment, the mixing device (1b) comprehends a body (2b) with cylindrical shape on the top part and the shape of an inverted cone body on the bottom part, with a central cylindrical channel (3b) and inclined downward channels (4b) that intercept the central cylindrical channel (3b) at its bottom end (5b). These inclined channels (4b) include a cylindrical recess (6b) at their top ends in order to receive a hermetic lid (60) by coupling fit.

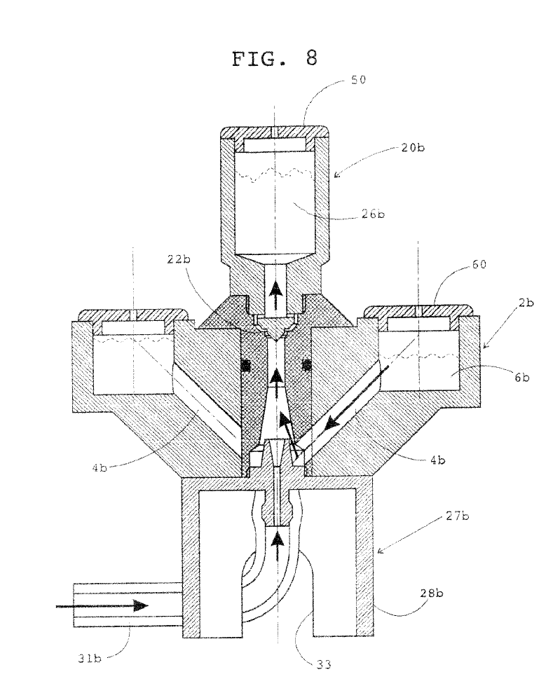

The mixing device (1b) also includes a central valve support (7b), a cylindrical base (9b), and a head (8b) in the shape of a cone. The base (9b) of the central support (7b) has a sealing ring (10b) on the outer surface and an internal opening (11b) inside it, which crosses the central valve support (7b) throughout its length. This opening (11b) has a cylindrical recess (12b) in its bottom end with an upward conical protrusion (13b) that reaches half the length of the central support (7b). Between this conical protrusion (13b) and the cylindrical recess (12b) there is a conical recess (14b) that includes an inclined through hole (15b), which enables the communication between the internal opening (11b) and the external cylindrical surface of the central support (7b). Above the conical protrusion (13b) there is a cylindrical channel (16b) that communicates with the head seat opening (17b), located in the region of the head (8b) of the central support (7b). This head seat opening (17b) includes conical housing recesses (18b) in which the conical tip of the head (21b) seats to form small passing openings (22b) (FIG. 8) for the foam with microbubbles.

The head (20b) is comprised of a cylindrical part with a tip (21b) of staggered conical shape that forms a step (23b) in its middle region to enable the formation of the passing channel (22b) of the foam with microbubbles (FIG. 8). Above the tip (21b) there are openings (24b) that communicate with the leer channel (25b) that receives the external part of the syringe to be fitted into the orifice (26b) of the head (20b). This opening also receives a hermetic lid (50).

At the bottom of the device (1b) a connecting base (27b) is connected and comprised of a cylindrical lid (28b) that is supported by the bottom part of the body (2b) and couples into the internal opening (11b) of the central valve support (7b) by means of a threaded protrusion (29b). Internally, this lid (28b) contains a fitting nozzle (30b) to receive a hose (31b), through which a gas enters and flows through the passage (32b) into the internal opening (11b) of the central valve support (7b). The cylindrical lid (28b) has a groove (33) on its side to receive the gas hose (31b).

The operation of the next device (1b) is similar to the description of the device above (1), differing only by the fact that, in lieu of syringes, the solutions are placed in small reservoirs (6b) contained in the body (2b). The foam formed with microbubbles is stored in the head (20b).

Therefore, as observed in the description detailed above, the device is comprised of a set of disassembleable and interchangeable parts that lock against each other for easy sterilization and handling, and resistance against extremely low or high temperatures. The device at hand is fully mounted on the body (2,2b) around an essentially cylindrical axle comprised of the central valve support (7,7b), while the top conical body portion comprehending the head (8,8b) is crossed in its center by a flow orienting duct (25, 25b). This axle, comprised of the central valve support (7,7b), is coupled at the center of a spinning circular reservoir constituted of the body (2,2b), containing several housings (6,26) for the application of several solutions with different concentration levels. These parts are locked by a connector (27) at the bottom to the gas duct (31) with a spray nozzle. Each housing (26) has a channel (4) that can be aligned to communicate with the inside of the flow orienting duct (11) of the central axle (7), responsible for orienting the produced mixture to a top reservoir with a foam-making nozzle.

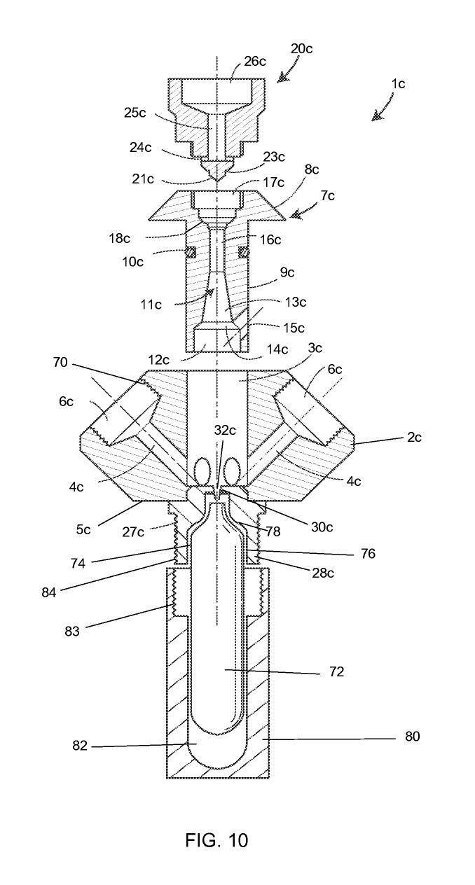

In the following embodiments, similar reference numerals are used to identify elements similar to the elements of embodiments of FIGS. 1-9. For example, the embodiment of FIG. 10 is a mixing device (1c) that is similar to the mixing device (1) of FIGS. 1 and 3-6 and to the mixing device (1b) of FIGS. 2 and 7-9. Subsequent embodiments include sequential letters (e.g., “c”, “d”, “e”, “f”) to designate similar features. Additionally, any feature or element (or combination of features or elements) described in one embodiment may be incorporated into any other embodiment.

FIG. 10 shows another embodiment of a rechargeable, resterilizable mixing device with physiological solution and gas to create foam with microbubbles used in endovascular treatments. In this embodiment, the mixing device (1c) includes a body (2c) with cylindrical shape on the top part and the shape of a truncated inverted cone body on the bottom part, with a central cylindrical channel (3c) and inclined downward channels (4c) that intercept the central cylindrical channel (3c) near a bottom end (5c). These inclined channels (4c) include a cylindrical recess (6c) at top ends in order to receive a solution reservoir (not shown in FIG. 10) via a connection, such as a threaded connection, for example, by internal threads (70) formed on an inner surface of the cylindrical recess (6c).

The mixing device (1c) also includes a central valve support (7c), a cylindrical base (9c), and a head (8c) in the shape of a cone. The base (9c) of the central support (7c) has a sealing ring (10c) on the outer surface and an internal opening (11c), which crosses the central valve support (7c) throughout its length. The internal opening (11c) has a cylindrical recess (12c) in a bottom end with an upward conical protrusion (13c) that reaches about half the length of the central support (7c). Between the conical protrusion (13c) and the cylindrical recess (12c) is a conical recess (14c) that includes an inclined through hole (15c), which enables fluid communication between the internal opening (11c) and the external cylindrical surface of the central support (7c). Above the conical protrusion (13c) is a cylindrical channel (16c) that fluidly communicates with the head seat opening (17c), located in the region of the head (8c) of the central support (7c). The head seat opening (17c) includes conical housing recesses (18c) in which the conical tip of the head (21c) seats to form small passing openings for the foam with microbubbles.

The head (20c) includes a cylindrical part with a tip (21c) of staggered conical shape that forms a step (23c) in a middle region to enable the formation of the passing channel of the foam with microbubbles. Above the tip (21c) are openings (24c) that fluidly communicate with the luer channel (25c), which receives an external part of a syringe to be fitted into the orifice (26c) of the head (20c).

At the bottom of the device (1c) a connecting base (27c) is connected and comprises a cylindrical sheath (28c) that is supported by the bottom part of the body (2c) and couples into the internal opening (11c) of the central valve support (7c). Internally, the cylindrical sheath (28c) contains a fitting nozzle (30c) to receive a reservoir of pressurized gas, such as a carbon dioxide cartridge 72, through which a gas enters and flows through a passage (32c) into the internal opening (11c) of the central valve support (7c). The cylindrical sheath (28c) may include a receiving recess (74) having a cylindrical portion (76) and a tapered portion (78) adapted to receive a neck of the pressurized gas cartridge (72). A connector housing (80) includes a cavity (82) that is sized and shaped to receive a body of the pressurized gas cartridge (72). The connector housing (80) may include internal threads (83) that cooperate with external threads (84) on the connecting base (27c) to releasably secure the connector housing (80) to the connecting base (27c).

The operation of the device (1c) is similar to the description of the devices above.

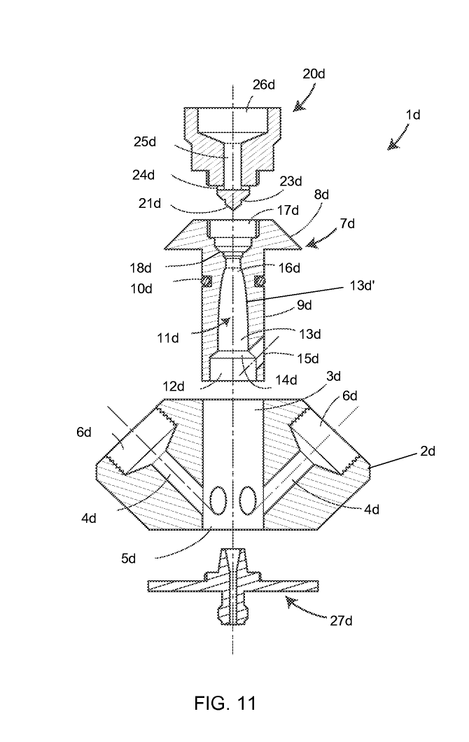

FIG. 11 shows another embodiment of the object of a rechargeable, resterilizable mixing device with physiological solution and gas to create foam with microbubbles used in endovascular treatments. In this embodiment, the mixing device (1d) includes a body (2d) with cylindrical shape on the top part and the shape of a truncated inverted cone body on the bottom part, with a central cylindrical channel (3d) and inclined downward channels (4d) that intercept the central cylindrical channel (3d) at a bottom end (5d). The inclined channels (4d) include a cylindrical recess (6d) at top ends in order to receive a solution reservoir (not shown in FIG. 11).

The mixing device (1d) also includes a central valve support (7d), a cylindrical base (9d), and a head (8d) in the shape of a cone. The base (9d) of the central support (7d) has a sealing ring (10d) on the outer surface and an internal opening (11d), which crosses the central valve support (7d) throughout its length. This opening (11d) has a cylindrical recess (12d) in its bottom end with an upward protrusion (13d) that reaches more than half the length of the central support (7d). Between the upward protrusion (13d) and the cylindrical recess (12d) is a conical recess (14d) that includes an inclined through hole (15d), which enables fluid communication between the internal opening (11d) and the external cylindrical surface of the central support (7d). Above the upward protrusion (13d) is a cylindrical channel (16d) that fluidly communicates with the head seat opening (17d), located in the region of the head (8d) of the central support (7d). The head seat opening (17d) includes conical housing recesses (18d) in which the conical tip of the head (21d) seats to form small passing openings for the foam with microbubbles.

The head (20d) includes a cylindrical part with a tip (21d) of staggered conical shape that forms a step (23d) in its middle region to enable the formation of the passing channel of the foam with microbubbles. Above the tip (21d) are openings (24d) that fluidly communicate with the luer channel (25d), which receives an external part of a syringe to be fitted into the orifice (26d) of the head (20d).

At the bottom of the device (1d) a connecting base (27d) may be included, which may be similar to the connecting base (27) of FIG. 6.

The embodiment of FIG. 11 differs from the first embodiment in the shape of the upward protrusion (13d). In the embodiment of FIG. 11, the upward protrusion (13d) has an internal wall having a reduction (13d′) in cross-sectional shape progressing from the conical recess (14d) towards the cylindrical channel (16d). In one preferred embodiment, the internal wall reduction (13d′) is an elliptical reduction. In other words, the internal wall reduction (13d′) has a partially elliptical shape when viewed in cross-section. The reduction (13d′) causes excellent mixing of the solution and gas to form microbubbles.

The operation of the device (1d) is similar to the description of the devices above.

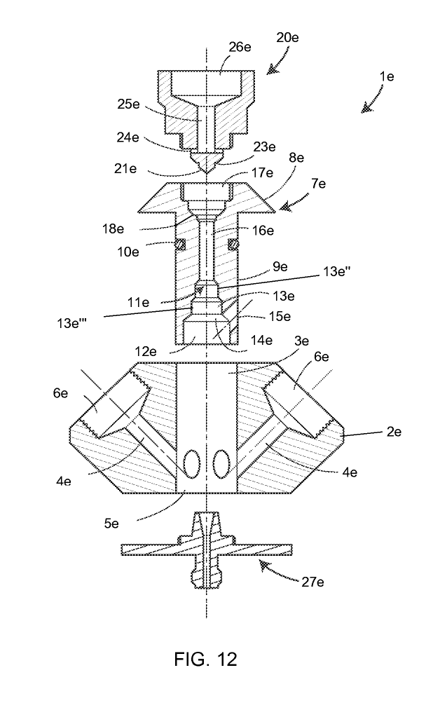

FIG. 12 shows another embodiment of the object of the rechargeable, resterilizable mixing device with physiological solution and gas to create foam with microbubbles used in endovascular treatments. In this embodiment, the mixing device (1e) includes a body (2e) with cylindrical shape on the top part and the shape of a truncated inverted cone body on the bottom part, with a central cylindrical channel (3e) and inclined downward channels (4e) that intercept the central cylindrical channel (3e) at its bottom end (5e). The inclined channels (4e) include a cylindrical recess (6e) at top ends to receive a solution reservoir (not shown in FIG. 12).

The mixing device (1e) also includes a central valve support (7e), a cylindrical base (9e), and a head (8e) in the shape of a cone. The base (9e) of the central support (7e) has a sealing ring (10e) on the outer surface and an internal opening (11e), which crosses the central valve support (7e) throughout its length. This opening (11e) has a cylindrical recess (12e) in its bottom end with an upward protrusion (13e) that reaches about half the length of the central support (7e). Between the upward protrusion (13e) and the cylindrical recess (12e) is a conical recess (14e) that includes an inclined through hole (15e), which enables fluid communication between the internal opening (11e) and the external cylindrical surface of the central support (7e). Above the upward protrusion (13e) is a cylindrical channel (16e) that communicates with the head seat opening (17e), located in the region of the head (8d) of the central support (7e). The head seat opening (17e) includes conical housing recesses (18e) in which the conical tip of the head (21e) seats to form small passing openings for the foam with microbubbles.

The head (20e) includes a cylindrical part with a tip (21e) of staggered conical shape that forms a step (23e) in a middle region to enable the formation of the passing channel of the foam with microbubbles. Above the tip (21e) are openings (24e) that fluidly communicate with the luer channel (25e), which receives an external part of a syringe to be fitted into the orifice (26e) of the head (20e).

At the bottom of the device (1e) a connecting base (27e) is included, which may be similar to the connecting base (27) of FIG. 6. The embodiment of FIG. 12 differs from the first embodiment in the shape of the upward protrusion (13e). In the embodiment of FIG. 12, the upward protrusion (13e) has an internal wall having a reduction progressing from the conical recess (14e) towards the cylindrical channel (16e). In one preferred embodiment, the reduction is a stepped reduction having a plurality of decreasing steps (13e″, 13e′″, etc.), which have decreasing internal radii. The reduction causes excellent mixing of the solution and gas to form microbubbles.

The operation of the device (1d) is similar to the description of the devices above.

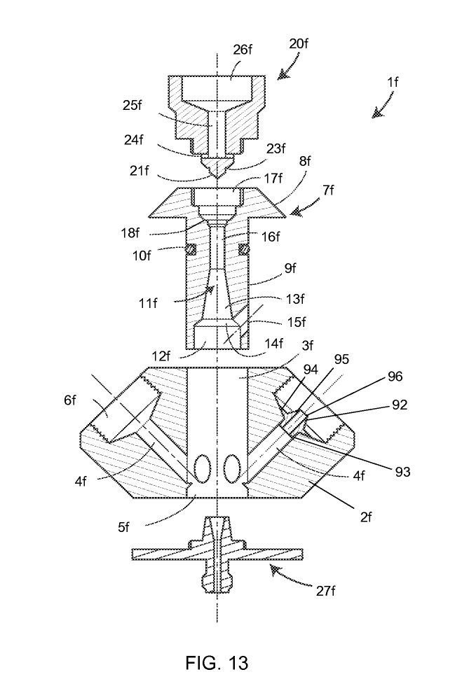

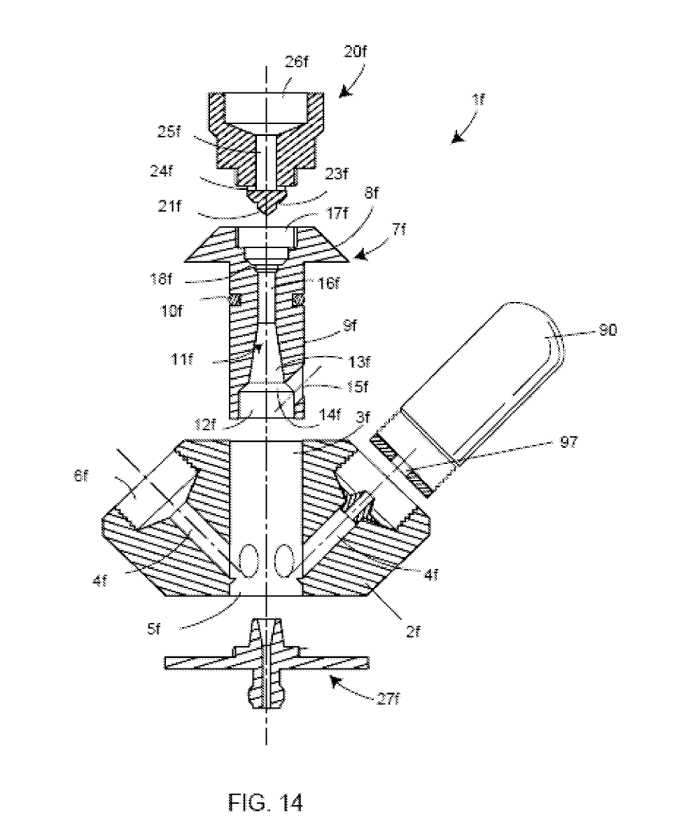

FIGS. 13 and 14 show another embodiment of the object of the rechargeable, resterilizable mixing device with physiological solution and gas to create foam with microbubbles used in endovascular treatments. In this embodiment, the mixing device (1f) includes a body (2f) with cylindrical shape on the top part and the shape of a truncated inverted cone body on the bottom part, with a central cylindrical channel (3f) and inclined downward channels (4f) that intercept the central cylindrical channel (3f) at its bottom end (5f). These inclined channels (4e) include a cylindrical recess (6f) at top ends in order to receive a solution reservoir 90 (FIG. 13).

The mixing device (1f) also includes a central valve support (7f), a cylindrical base (9f), and a head (8f) in the shape of a cone. The base (9f) of the central support (7f) has a sealing ring (10f) on an outer surface and an internal opening (11f), which crosses the central valve support (7f) throughout its length. The opening (11f) has a cylindrical recess (12f) in a bottom end with an upward protrusion (13f) that reaches about half the length of the central support (7f). Between the upward protrusion (13f) and the cylindrical recess (12f) is a conical recess (14f) that includes an inclined through hole (15f), which enables fluid communication between the internal opening (11f) and the external cylindrical surface of the central support (7f). Above the upward protrusion (13f) is a cylindrical channel (16f) that fluidly communicates with the head seat opening (17f), located in the region of the head (8f) of the central support (7f). The head seat opening (17f) includes conical housing recesses (18f) in which the conical tip of the head (21f) seats to form small passing openings for the foam with microbubbles.

The head (20f) includes a cylindrical part with a tip (21f) of staggered conical shape that forms a step (23f) in a middle region to enable the formation of the passing channel of the foam with microbubbles. Above the tip (21f) are openings (24f) that fluidly communicate with the luer channel (25f), which receives the external part of the syringe to be fitted into the orifice (26f) of the head (20f).

At the bottom of the device (1f) a connecting base (27e) may be included, which may be similar to the connecting base (27) of FIG. 6.

The embodiment of FIGS. 13 and 14 includes a solution fitting (92) disposed within the cylindrical recess (6f). In one embodiment, the solution fitting (92) may be an integral component of the base (2f) and in other embodiments, such as the embodiment illustrated in FIGS. 13 and 14, the solution fitting (92) may be a separate component. The solution fitting may include a cylindrical base (93) that fits within the inclined channel (4f). An outwardly extending flange (94) may be located between the cylindrical base (93) and a nozzle (95). The outwardly extending flange (94) seats within the cylindrical recess (6f) to prevent the solution fitting (92) from being pushed into the inclined channel (4f). The outwardly extending flange (94) also stabilizes the solution fitting within the cylindrical recess (6f). A central bore (96) fluidly connects the cylindrical recess (6f) with the inclined channel (4f). The nozzle (95) cooperates with an opening (97) in the solution reservoir (90) to allow solution from the solution reservoir (90) to pass into the inclined channel (4f). The solution fitting (92) allows rapid fitting and replacing of the solution reservoir (90).

The operation of the device (1f) is similar to the description of the devices above.

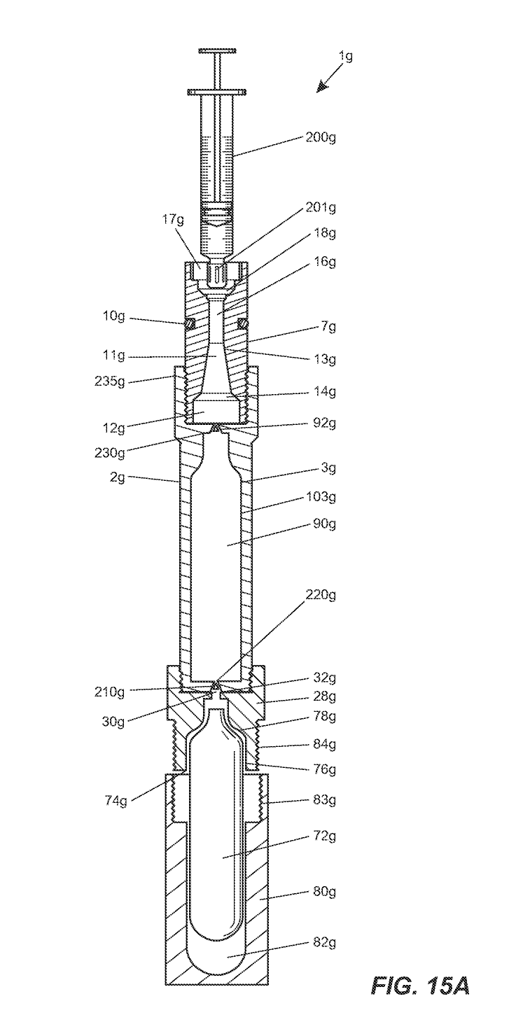

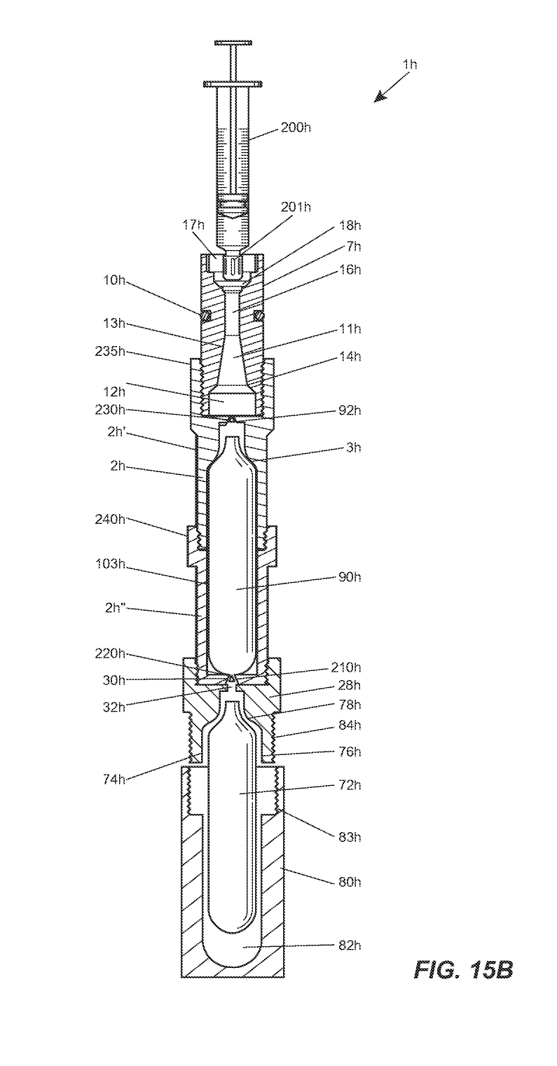

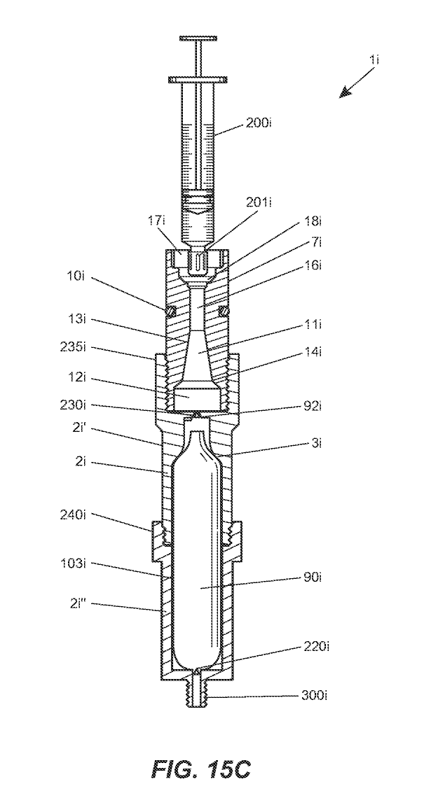

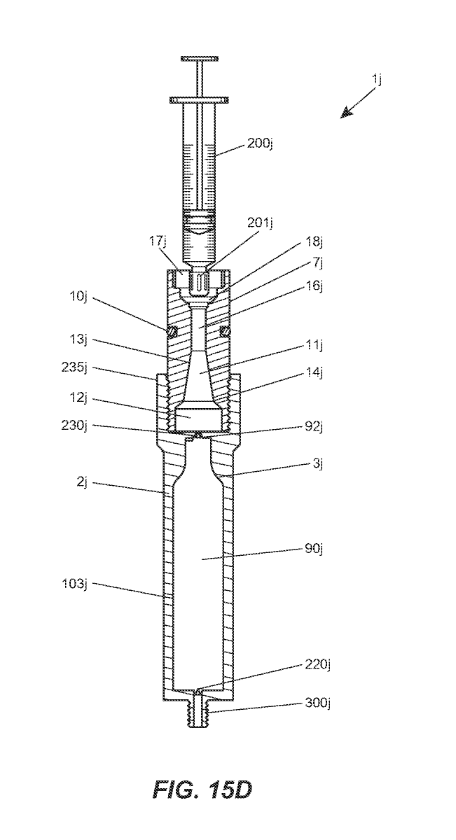

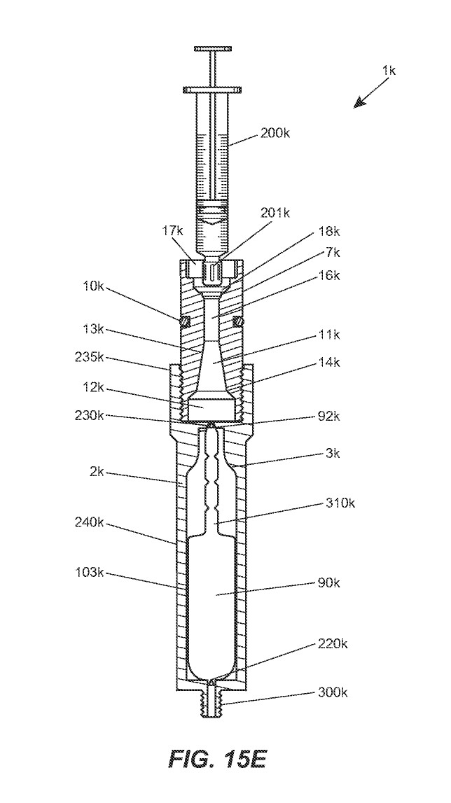

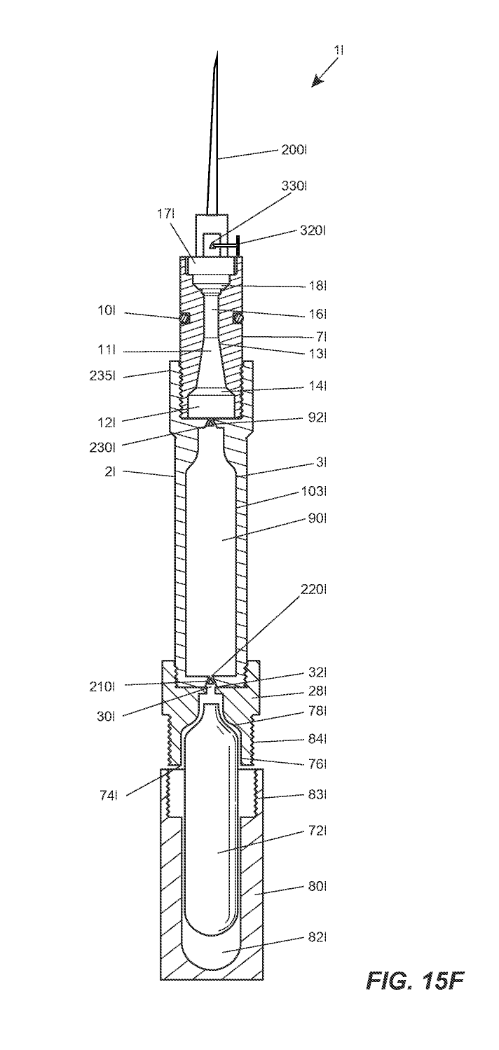

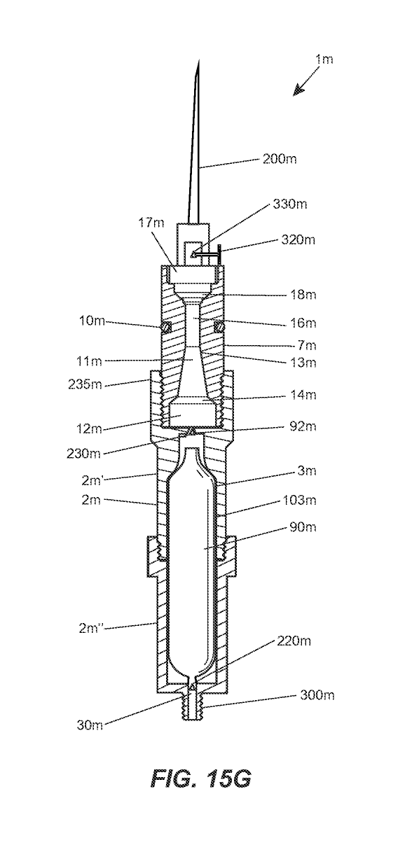

FIGS. 15A and 15B show other embodiments of the object of the rechargeable, resterilizable mixing device with physiological solution and gas to create foam with microbubbles used in endovascular treatments. In these embodiments, the mixing device (1g, 1 h) includes a body (2g, 2h) having a cylindrical shape, with a central cylindrical channel (3g, 3h). The embodiments of FIGS. 15A and 15 B differ from previous embodiments in that the embodiments of FIGS. 15A and 15B do not include any inclined downward channels that intercept the central cylindrical channel (3g, 3h). Rather, the central cylindrical channels (3g, 3h) form a chamber (103g, 103h) that comprises a reservoir (90g) of foaming solution or a cartridge (90h) of foaming solution.

The mixing device (1g, 1h) also includes a central syringe support (7g, 7h). The central syringe support (7g, 7h) has a sealing ring (10g, 10h) on an outer surface and an internal opening (11g, 11h), which crosses the central syringe support (7g, 7h) throughout its length. The opening (11g, 11h) has a cylindrical recess (12g, 12h) in a bottom end with an upward protrusion (13g, 13h) that reaches about half the length of the central syringe support (7g, 7h). Between the upward protrusion (13g, 13h) and the cylindrical recess (12g, 12h) is a conical recess (14g, 14h), which provides fluid communication between the internal opening (11g, 11h) and the cylindrical recess (12g, 12h). Above the upward protrusion (13g, 13h) is a cylindrical channel (16g, 16h) that fluidly communicates with a syringe seat opening (17g, 17h), located in one end of the central syringe support (7g, 7h). The syringe seat opening (17g, 17h) includes a conical recess (18g, 18h) in which the tip of a syringe (200g, 200h) seats to form small passing openings for the foam with microbubbles.

The internal part of the syringe (200g, 200h) to be fitted into the conical recess (18g, 18h may include a plurality of channels or recesses (201g, 201 h) that enhance the formation of microbubbles in the foam.

The embodiments of FIGS. 15A and 15B include a cylindrical sheath (28g, 28h) that is supported by the bottom part of the body (2g, 2h) and couples to an opening (210g, 210h) in the bottom of the body (2g, 2h). Internally, the cylindrical sheath (28g, 28h) contains a fitting nozzle (30g, 30h) and a one-way check valve (220g, 220h) to receive a reservoir of pressurized gas, such as a carbon dioxide cartridge (72g, 72h), through which a gas enters and flows through a passage (32g, 32h) and through the one way check valve (220g, 220h) into the central cylindrical channel (3h, 3h) of the body (2g, 2h). The cylindrical sheath (28g, 28h) may include a receiving recess (74g, 74h) having a cylindrical portion (76g, 76h) and a tapered portion (78g, 78h) adapted to receive a neck of the pressurized gas cartridge (72g, 72h). A connector housing (80g, 80h) includes a cavity (82g, 82h) that is sized and shaped to receive a body of the pressurized gas cartridge (72g, 72h). The connector housing (80g, 80h) may include internal threads (83g, 83h) that cooperate with external threads (84g, 84h) on the cylindrical sheath (28g, 28h) to releasably secure the connector housing (80g, 80h) to the cylindrical sheath (28g, 28h).

The body (2g, 2h) may include a solution fitting (92g, 92h) disposed within the central cylindrical channel (3g, 3h). In one embodiment, the solution fitting (92g, 92h) may be an integral component of the body (2g, 2h) and in other embodiments, the solution fitting (92g, 92h) may be a separate component. The solution fitting (92g, 92h) may include a one-way check valve (230g, 230h) that allows solution and gas to flow in only one direction, from the reservoir (103g) or cartridge (103h) towards the central syringe support (7g, 7h). The body (2g, 2h) may also include a collar (235g, 235h) that threadedly engages the central syringe support (7g, 7h).

One difference between the embodiment of FIG. 15A and the embodiment of FIG. 15B is that the body (2h) of the embodiment of FIG. 15B is formed in two releasably connected sections (2h′, 2h″). One of the sections (2h′, 2h″) may include a collar (240h) that threadedly engages the other of the sections (2h′, 2h″) so that the two sections (2h′ and 2h″) may be separated from one another to facilitate replacement of the foam cartridge (90h).

The operation of the devices (1g, 1 h) is similar to the description of the devices above.

FIGS. 15C-15G illustrate other embodiments of the device (1i, 1j, 1k, 11, 1m), where like elements have like reference numerals, the only difference being the letter suffix. For example, the embodiment illustrated in FIG. 15C includes a body (2i) and a central syringe support (7i). The embodiment of FIG. 15C differs from other embodiments in that the physiological gas is introduced directly into the cartridge (90i) of foaming solution through a fitting (300i). A one-way check valve (220h) controls flow of the physiological gas into the cartridge (90i). Additionally, the body (2i) is formed in two parts (2i′, 2i″) for easy access to the cartridge (90i) so that the cartridge (90i) may be replaced and the device (1i) may be reused.

Similarly, the embodiment illustrated in FIG. 15D is similar to the embodiment illustrated in FIG. 15C except that the body (2j) forms a reservoir (90j). Physiological gas is introduced into the reservoir (90j) through a one-way check valve (220j). The device (1j) illustrated in FIG. 15D may be a disposable device, at least as far as the body (2j) is concerned, because the body (2j) is formed as a single piece disposable unit.

The embodiment illustrated in FIG. 15E may include a single piece disposable body (2k), similar to the body of FIG. 15D. However, the reservoir (90k) may include a channel (310k) having a varying diameter. The channel (310k) may enhance mixing of the physiological gas and the sclerosing agent.

The embodiment of FIG. 15F includes an injection needle (2001) instead of a syringe. The injection needle (2001) may be removably attached to the central syringe support (71), which results in a reusable device (11), or the injection needle (2001) may be fixed to the central syringe support (71), which results in a disposable device (11). An actuating lever (3201) may be operatively connected to a control valve (3301), which is disposed between the injection needle (2001) and the central syringe support (71) to control flow of the mixed physiological gas and sclerosing agent into a patient.

The embodiment of FIG. 15G may be similar to the embodiment of FIG. 15F except that the embodiment of FIG. 15G may include a two-piece body (2m) and a cartridge (90m). The two pieces (2m′, 2m″) may be separated to replace the cartridge (90m), which produces a reusable device (1m).

While many different types of solutions may be used in the disclosed mixing device, one preferred embodiment uses a solution reservoir (or a syringe) containing polidocanol to mix with a pressurized gas, such as ambient air or carbon dioxide, to produce a foam including microbubbles. In yet other embodiments, the body of the mixing device may include self-contained reservoirs of mixing solution.

Although certain mixing devices have been described herein in accordance with the teachings of the present disclosure, the scope of coverage of this patent is not limited thereto. On the contrary, while the invention has been shown and described in connection with various preferred embodiments, it is apparent that certain changes and modifications, in addition to those mentioned above, may be made. This patent covers all embodiments of the teachings of the disclosure that fairly fall within the scope of permissible equivalents. Accordingly, it is the intention to protect all variations and modifications that may occur to one of ordinary skill in the art.

1. Rechargeable, resterilizable mixing device for mixing a physiological solution and a gas to create foam with microbubbles for use in endovascular treatments, the mixing device comprising a body in the shape of two inverted cones united by their bases, with a central cylindrical channel and inclined downward channels that intercept the central cylindrical channel at a bottom end of the body, the inclined downward channels containing a cylindrical recess at a top end, the mixing device further including a central valve support having a cylindrical base and a cone-shaped head, the cylindrical base of the central valve support including a sealing ring on an external surface and including an internal opening crossing a length of the central valve support, the internal opening having a cylindrical recess on a bottom end of the central valve support with a conical protrusion extending upward until a middle region of the length of the central valve support, the internal opening including a conical recess disposed between the conical protrusion and the cylindrical recess, the conical recess containing an inclined passage hole that enables communication between the internal opening and the external cylindrical surface of the central valve support, a cylindrical channel is disposed above the conical protrusion and communicates with an opening of a head seat, located in a region of the cone-shaped head of the central valve support, the opening of the head seat contains conical housing recesses to receive a conical tip of a head piece thereby forming small passage openings the head piece including a cylindrical part with a staggered conical tip that forms a step adjacent a middle and enabling the formation of the small passage, openings are included above the tip that communicate with a luer channel, adjacent a bottom part of the mixing device there is a coupled connector comprising a thin cylindrical disk supported by the bottom end of the body and coupled with the internal opening of the central valve support by means of a threaded protrusion the coupled connector including a fitting nozzle to receive a hose.

2. Rechargeable, resterilizable mixing device for mixing a physiological solution and a gas to create foam with microbubbles for use in endovascular treatments, the mixing device comprising a body with cylindrical shape on a top part and the shape of an inverted cone body on a bottom part, the mixing device including a central cylindrical channel and inclined downward channels that intercept the central cylindrical channel at a bottom end of the central cylindrical channel, the inclined downward channels include a cylindrical recess at their top ends in order to receive a hermetic lid, the mixing device also including a central valve support having a cylindrical base and a head in the shape of a cone, the cylindrical base of the central valve support including a sealing ring on an external surface of the central valve support and an internal opening which crosses a length of the central valve support, the opening including a cylindrical recess in a bottom end of the central valve support with an upward conical protrusion that reaches half the length of the central valve support, a conical recess is disposed between the conical protrusion and the cylindrical recess, the conical recess containing an inclined passage hole, above the conical protrusion there is a cylindrical channel that communicates with a head seat opening located in a region of the head of the central valve support, the head seat opening including conical housing recesses in which a conical tip of a head piece seats to form small passing openings the head piece being comprised of a cylindrical part with the conical tip having a staggered conical shape that forms a step in its middle region, above the tip there are openings that communicate with a luer channel, adjacent a bottom of the device a connecting base is connected, the connecting base including a cylindrical lid that is supported by the bottom part of the body and couples into the internal opening of the central valve support by means of a threaded protrusion, the lid including a fitting nozzle and a side groove.

3. A device for creation of endovascular foam, the device comprising:
a body, the body having a central channel and a plurality of peripheral channels, the central channel having a bottom end, each of the peripheral channels intersecting the central channel adjacent the bottom end of the central channel, a second end of each of the peripheral channels including an upper recess;
a central valve support coupled to the body, the central valve support including a base having an external surface sized for placement in the central channel of the body, the central valve support further including an internal opening extending lengthwise through the central valve support and a passage hole extending between the internal opening and the external surface of the base, the passage hole arranged to provide flow communication between the internal opening and the external surface of the base, the central valve support further including a head seat and an upper recess, the upper recess and the head seat in flow communication with the internal opening;
a head arranged for attachment to the head seat of the central valve support, the head having a tip, a channel, and openings in communication with the channel, the tip sized for placement in the recess of the central valve support, the tip of the head cooperating with the recess of the central valve support to form a passing channel;
a coupled connector coupled adjacent a bottom part of the body, the coupled connector including a passage in flow communication with the internal opening of the central valve support and including a fitting arranged to receive a hose; and
wherein the body is arranged for rotation relative to the base to bring a selected one of the peripheral channels into flow communication with the internal opening of the central valve support via the passage hole.

4. The device of claim 3, wherein at least a portion of the internal opening of the central valve support is conical.

5. The device of claim 3, wherein the internal opening of the central valve support includes a conical recess adjacent to a conical protrusion.

6. The device of claim 4, wherein the conical recess of the internal opening is disposed adjacent the passage hole.

7. The device of claim 4, wherein the conical protrusion of the internal opening extends adjacent a middle region of the central valve support.

8. The device of claim 3, wherein the tip of the head is staggered to form a step in the passing channel.

9. The device of claim 8, wherein the tip of the head and the upper recess of the head are conical.

10. The device of claim 3, wherein the head includes an orifice, the upper recess of each of the peripheral channels is arranged to receive a physiological solution, and wherein the orifice of the head is arranged to receive the endovascular foam.

11. The device of claim 10, wherein the upper recess of each of the peripheral channels comprises a reservoir arranged to receive the physiological solution.

12. The device of claim 10, wherein the upper recess of each of the peripheral channels is arranged to receive a syringe body containing the physiological solution.

13. The device of claim 11, wherein the upper recess of at least one of the peripheral channels comprises internal threads.

14. The device of claim 7, wherein the conical protrusion includes a reduction bore.

15. The device of claim 14, wherein the reduction bore has an elliptical cross-sectional shape.

16. The device of claim 14, wherein the reduction bore comprises a plurality of decreasing radius steps.

17. The device of claim 3, wherein the upper recess of at least one peripheral bore includes a solution fitting.

18. The device of claim 17, wherein the solution fitting includes a cylindrical base that fits within the at least one peripheral bore.

19. The device of claim 18, wherein the solution fitting further includes an outwardly extending flange disposed between the cylindrical base and a nozzle.

20. The device of claim 19, wherein the solution fitting further includes a central bore.

21. A device for creation of endovascular foam, the device comprising:
a body, the body having a central channel, the central channel having an opening in a bottom end and a one-way check valve at a top end;
a central syringe support coupled to the body, the central syringe support further including an internal opening extending lengthwise through the central syringe support, the central valve support further including an upper conical recess, the upper conical recess being in flow communication with the internal opening; and
a cylindrical sheath support by a bottom part of the body and coupled to an opening in the bottom part of the body, the cylindrical sheath including a fitting nozzle and a one-way check valve near a top of the cylindrical sheath, the one-way check valve allowing fluid flow in one direction only, from the cylindrical sheath into the body.

22. The device of claim 21, further comprising a gas cartridge disposed in the cylindrical sheath.

23. The device of claim 21, further comprising a foam solution cartridge in the body.

24. The device of claim 23, wherein the body comprises to removably attached pieces that may be separated from one another to expose an internal chamber for changing the foam cartridge.

25. The device of claim 21, further comprising an injection needle removably attached to the central syringe support.

26. A device for creation of endovascular foam, the device comprising:
a body, the body having a central channel, the central channel having an opening in a bottom end and a one-way check valve at a top end and a physiological gas fitting at a bottom end for the introduction of a physiological gas into the central channel; and
a central syringe support coupled to the body, the central syringe support further including an internal opening extending lengthwise through the central syringe support, the central valve support further including an upper conical recess, the upper conical recess being in flow communication with the internal opening.

27. The device of claim 26, wherein the body is formed from two removably attached pieces, the two pieces being separable from one another to expose the central channel.

28. The device of claim 26, further comprising a foam solution cartridge disposed within the body, the foam solution cartridge being removable from the body when the two pieces are separated from one another.

29. The device of claim 26, wherein the body includes a reservoir having a channel with a varying diameter.

30. The device of claim 26, further comprising an injection needle removably attached to the central syringe support.

31. The device of claim 26, wherein the physiological gas fitting includes external threads.
